(19) United States
(12) Patent Application Publication
(10) Pub. No.: US 2012/0172913 A1
(43) Pub. Date: Jul. 5, 2012

(54) DELIVERY OF AN EMBOLIZATION COIL WITH AN ATTACHER

(75) Inventors: Michael R. Kurrus, Ellettsville, IN (US); William F. Moore, Bloomington, IN (US); Jeffry S. Melsheimer, Springville, IN (US); Kathryn R. Evert, Bloomington, IN (US)

(73) Assignee: Cook Medical Technologies LLC, Bloomington, IN (US)

(21) Appl. No.: 13/338,739

(22) Filed: Dec. 28, 2011

Related U.S. Application Data
(60) Provisional application No. 61/428,420, filed on Dec. 30, 2010.

(51) Int. Cl. A61F 2/01

(52) U.S. Cl. 606/200

(57) ABSTRACT

An occluding device apparatus includes an embolization coil with a distal end and a proximal end with an opening and an attacher that is threaded through the opening at the proximal end of the embolization coil. The apparatus further comprises a delivery kit for delivery of the embolization coil in a body cavity. The kit comprises a guide catheter for percutaneous introduction of the embolization coil and an inner catheter slidably disposed within the guide catheter during insertion. The inner catheter comprises a proximal end and a distal end. The inner catheter further includes a hub disposed adjacent the proximal end. The kit further comprises a guide wire slidably disposed within the inner catheter. The guide wire is configured to provide a path during insertion thereof within a body cavity. The kit further comprises a pushwire to advance the embolization coil through the inner catheter.

### CROSS-REFERENCE TO RELATED APPLICATIONS

This application claims the benefit of U.S. Provisional Application No. 61/428,420, filed on Dec. 30, 2011, entitled “DELIVERY OF AN EMBOLIZATION COIL WITH AN ATTACHER,” the entire contents of which are incorporated herein by reference.

### BACKGROUND OF THE INVENTION

1. Field of Invention

The present invention relates to medical devices. More particularly, the invention relates to occluding devices and methods of occluding fluid flow through a body vessel.

2. Background

Embolization coils have been used as a primary occluding device for treatment of various arteriovenous malformations (AVM) and varicoceles, as well as for many other arteriovenous abnormalities in the body. Occluding devices are also used to repair abnormal shunts between arteries and veins, prevent or reduce blood flow to tumors, stop hemorrhaging as a result of trauma, and stabilize aneurysms to prevent rupture. Embolization coils, for example pushable fibered coils, may be configured in a variety of sizes with varying diameters and may be made of several different materials including stainless steel and platinum. Occlusion devices may vary for differing purposes, e.g., to hold the device in place within a cavity or vessel and to pack the device within the vessel for enhanced occlusion.

Although current coils are adequate, such coils may be improved for more effective occlusion of fluid flow through a lumen of a body vessel. Many medical procedures for occluding blood flow through an artery or vein require a number of coils, since a single coil or two may not be sufficient to effectively occlude blood flow through a lumen of an artery or vein. For example, a coil having greater stiffness or rigidity may be introduced into a blood vessel and various coils of decreasing stiffness or rigidity may follow behind the stiffer coil. This procedure may involve an undesirable amount of additional time and increased costs associated with manufacturing and deploying a number of different coils.

### BRIEF SUMMARY OF THE INVENTION

The present invention provides an improved occluding device and an improved method of delivering the device for occluding fluid flow through a lumen of a body vessel.

In one form, the occluding device includes an embolization coil with a distal end and a proximal end with an opening and a suture or an attacher that is threaded through the opening at the proximal end of the embolization coil.

In another form, the occluding device includes an embolization coil with a distal end and a proximal end and a suture or an attacher that is tied as a slip-knot around the proximal end of the embolization coil.

Other forms of the invention includes methods of using the aforementioned occlusion devices to occlude fluid flow in a body vessel.

Further features and advantages will become apparent from the following description and from the claims.

### BRIEF DESCRIPTION OF THE DRAWINGS

FIG. 1a is a partial side view of a coiled wire in accordance with one embodiment of the present invention;

FIG. 1b is a partial side perspective view of an occluding device in accordance with the embodiment of FIG. 1a;

FIG. 2 is a cross-sectional environmental view of an occluding device deployed in a body vessel;

FIG. 3 is a close up view of a suture delivery arrangement for an occluding device in accordance with an embodiment of the present invention;

FIG. 4 is a close up view of a suture delivery arrangement for an occluding device in accordance with another embodiment of the present invention;

FIG. 5a is a close up view of a suture delivery arrangement for an occluding device in accordance with yet another embodiment of the present invention;

FIG. 5b is a close up view of a slip knot for the suture delivery arrangement of FIG. 5a;

FIG. 6a is an exploded view of an embolization kit in accordance with an embodiment of an occluding device of the present invention;

FIG. 6b is a side view of an embolization kit in accordance with an embodiment of the present invention;

FIG. 7 is a flowchart for delivering an occluding device with a suture delivery arrangement in accordance with an embodiment of the present invention; and

FIG. 8 is a flowchart for delivering an occluding device with a suture delivery arrangement in accordance with yet another embodiment of the present invention

### DETAILED DESCRIPTION OF THE INVENTION

The following provides a detailed description of currently preferred embodiments of the present invention. The description is not intended to limit the invention in any manner, but rather serves to enable those skilled in the art to make and use the invention.

The present invention generally provides an occluding device, and delivery for such a device, used for transcatheter embolization to provide an improved occlusion of fluid flow through the vessel. The occluding device is an embolization coil preferably used to occlude fluid flow through a lumen of a body vessel such as for an occlusion of an arteriovenous malformation (AVM). The occluding device include a primary coil formed into a helical shape and further defines a secondary coil. To further facilitate occlusion of fluid flow the occluding device may include fibers attached between loops of the primary coil and extending therefrom.

The occluding device also may be employed for treatment of renal arteriovenous malfunction (AVM), pulmonary AVM, vascular tumors, low-flow fistulas, trauma related hemorrhages, and visceral vasculature defects including varicoceles, aneurysms, and selected telangiectasias. For example, treatment of visceral vasculature defects may include but are not limited to embolotherapy on gastroduogenal hemorrhages, hepatic aneurysms, celiac aneurysms, internal iliac aneurysms, and internal spermatic varicoceles.

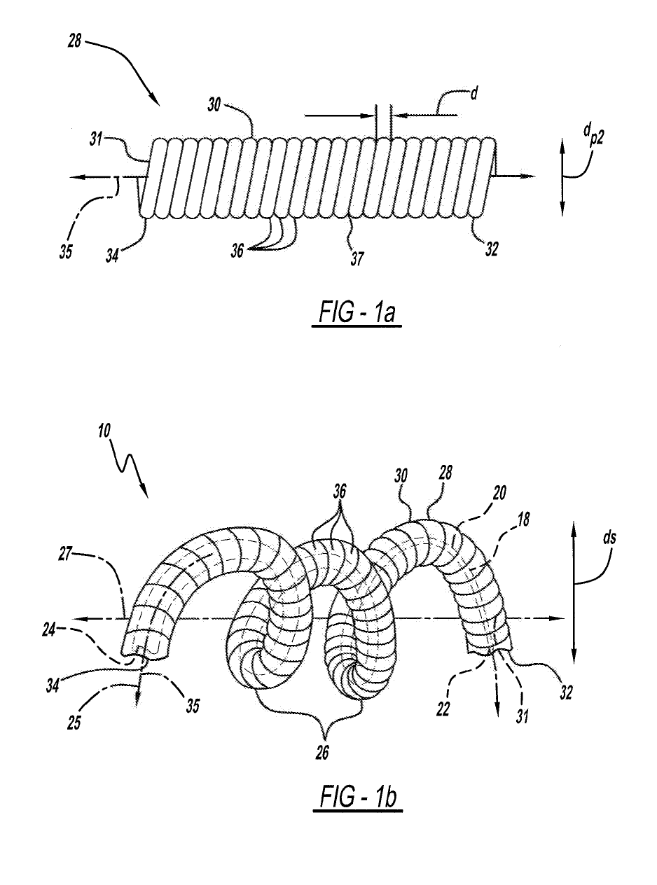

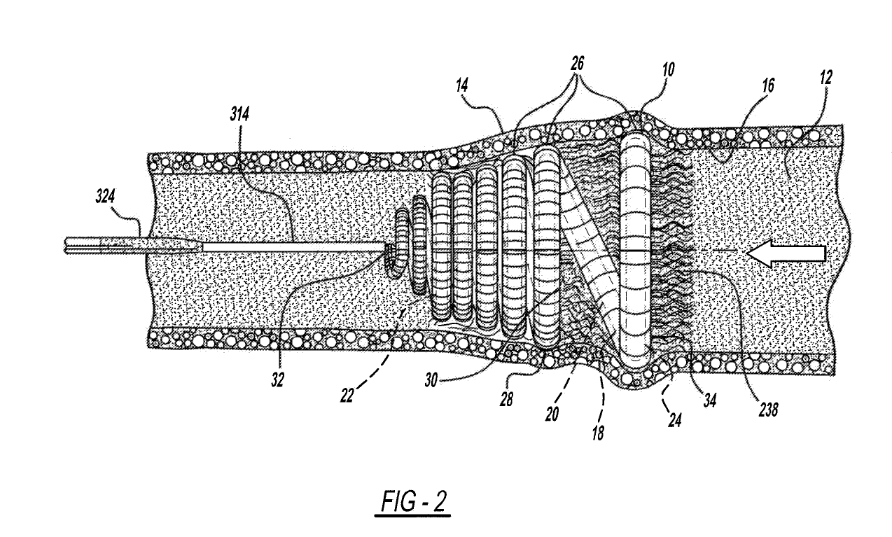

Referring to FIGS. 1 through 2, various embodiments of an occluding device in accordance with the present invention are shown. FIG. 2 illustrates an occluding device 10 such as an embolization coil in a deployed state for occlusion of fluid flow through a lumen 12 of a body vessel 14. As shown, the occluding device 10 is positioned to engage an inner wall 16 of the body vessel 14 and includes a primary coil 18 and a secondary coil 28.

As illustrated in FIG. 1a, a wire 30 having a proximal end 32 and a distal end 34 is wound about a longitudinal axis 35 into a secondary coil 28 having proximal and distal ends 32, 34, respectively. The longitudinal axis 35 forms the central axis of the secondary coil 28. In this embodiment, the wire 30 has a generally constant diameter and thus the secondary coil 28 has a generally constant diameter d, shown in FIG. 1a.

Typically, the wire 30 is wound about the longitudinal axis 35 into a longitudinally extending secondary coil 28 having an inner lumen 31 through which a wire 20 (FIG. 1b) may extend. The wire 20 includes a proximal end 22, a distal end 24, and a central axis 25 extending between the proximal and distal ends 22, 24. The wire 20 may be tapered along the central axis 25 or it may have a constant diameter.

In some implementations, the wire 20 is curled or coiled about a longitudinal axis 27 into a primary coil 18 having a primary shape defined by a plurality of turns or loops 26 wound about the longitudinal axis 26 of the primary coil 18 and axially spaced apart by a predetermined distance. The plurality of loops 26 defines a cross-sectional area formed axially along the primary coil 18. The wire 20 may be coiled into the primary coil 18 by any apparatus known in the art, such as a roller deflecting apparatus, a mandrel apparatus, or any other suitable means. For example, the wire 20 may be wound about a mandrel and heat set to form its spiral shape. Alternatively, the wire 20 may be wound about a longitudinally tapered mandrel and heat set to form a conically helically shaped coil.

In this embodiment, the secondary coil 28 has a generally linear primary shape and includes a plurality of tightly spaced turns 36 with minimal, if any spacing 37 therebetween. The generally linear primary shape is defined by a generally constant primary diameter dₚ₂. The wire 30 is wound into the secondary coil 28 by any apparatus known in the art, such as a roller deflecting apparatus, a mandrel apparatus, or any other suitable means. For example, the wire 30 may be wrapped around a mandrel and heat set to form its primary shape.

As illustrated in FIG. 1b, the wire 20 is received within the lumen 31 of the secondary coil 28, wherein the coiled wire 20 (i.e., the primary coil 18) provides the secondary coil 28 with its secondary shape. The wire 20 is initially curled or coiled into the primary coil 18. In one embodiment, the linear longitudinally extending secondary coil 28 is threaded or slid over the wire 20 in its coiled configuration (i.e., the primary coil 18).

In this embodiment, the central axis 35 of the secondary coil 28 is aligned with the central axis 25 of the coiled wire 20. With the distal end 34 of the secondary coil 28 adjacent the proximal end 22 of the coiled wire 20, the secondary coil 28 slides over the coiled wire 20 until the distal end 34 of the secondary coil 28 meets the distal end 24 of the coiled wire 20. In this arrangement, the secondary coil 28 conforms to the shape of the coiled wire 20 as the overlying secondary coil 28 moves along the plurality of loops 26 of the coiled wire 20, coiling about the longitudinal axis 27, and thus forming the secondary shape of the secondary coil 28.

In another arrangement, the coiled wire 20 may be straightened before being received within the lumen 31 of the linear longitudinally extending secondary coil 28. In this arrangement, the central axis 35 of the secondary coil 28 is aligned with the central axis 25 of the wire 20. With the distal end 34 of the secondary coil 28 adjacent the proximal end 22 of the wire 20, the secondary coil 28 slides over the straightened wire 20 until the distal end 34 of the secondary coil 28 meets the distal end 24 of the tapered wire 20. Thereafter, the wire 20 within the secondary coil 28 returns to its coiled configuration (i.e., the primary coil 18) causing the secondary coil 28 to take the shape of the primary coil 18, both the primary coil 18 and the secondary coil 28 coiling about the longitudinal axis 27, thus forming the secondary shape of the secondary coil 28.

Thus, the coiled wire 20 (i.e., primary coil 18) provides the secondary coil 28 with its secondary shape defined by the plurality of axially spaced loops 26. Thus, the wire 20 serves as an inner mandrel within the secondary coil 28. If the wire 20 is tapered it further provides the secondary coil 28 with a gradually decreasing stiffness from the distal end 34 to the proximal end 32, resulting in a variable strength occluding device.

Further details of the aforementioned occluding devices are described in U.S. application Ser. No. 12/171,900, filed Jul. 11, 2008, the entire contents of which are incorporated herein by reference.

The secondary shape of the secondary coil 28 is shaped by the primary shape of the primary coil 18, and thus the secondary diameter dₛ corresponds with the primary diameter of the primary coil 18 and may be generally constant or varied. Alternatively, the secondary shape may be non-linear and include a plurality of radially expanding loops 26 (i.e., a radially increasing secondary diameter dₛ) forming a conically helically shaped coil, an example of which is illustrated in FIG. 2.

As shown in FIG. 2, to assist in occluding fluid flow through the lumen 12 of the body vessel 14, the occluding device 10 may further include a series of fibers 238 attached between loops 26 of the secondary coil 28 and extending therefrom. Note, in this particular implementation, the wire 30 has a tapered diameter from the distal end 34 to the proximal end 32. Accordingly, the wire 30 has a gradually or continuously decreasing diameter from the distal end 34 to the proximal end 32 such that every successive point along the secondary coil 28 proximal the distal end 34 has a diameter successively smaller than the diameter at the distal end 34 and every successive point along the secondary coil.

The fibers 238 may be attached to the wire 30 before or after the wire 30 is coiled into the secondary coil 28. In a preferred embodiment, the fibers 238 include strands comprising a synthetic polymer such as polyester textile fiber, e.g., DACRON™. As desired, the strands may be positioned between adjacent loops, alternating loops, alternating double loops, or any desired configuration.

Preferably, the wires 20, 30 making up the primary 18 and secondary coils 28 are made of any suitable material that will result in the device 10 capable of being percutaneously inserted and deployed within a body cavity. Examples of preferred materials include metallic materials, such as stainless steel, platinum, iron, iridium, palladium, tungsten, gold, rhodium, rhenium, and the like, as well as alloys of these metals. Other suitable materials include superelastic materials, a cobalt-chromium-nickel-molybdenum-iron alloy, a cobalt chrome-alloy, stress relieved metal, nickel-based superalloys, such as Inconel, or any magnetic resonance imaging (MRI) compatible material, including materials such as a polypropylene, nitinol, titanium, copper, or other metals that do not disturb MRI images adversely. The wires 20, 30 may also be made of radiopaque material, including tantalum, barium sulfate, tungsten carbide, bismuth oxide, barium sulfate, and cobalt alloys.

Further, the wires 20, 30 making up the primary 18 and secondary coils 28 may be fabricated from shape memory materials or alloys, such as superelastic nickel-titanium alloys. An example of a suitable superelastic nickel-titanium alloy is Nitinol, which can “remember” and recover a previous shape. Nitinol undergoes a reversible phase transformation between a martensitic phase and an austenitic phase that allows it to “remember” and return to a previous shape or configuration. For example, compressive strain imparted to the coils 18, 28 in the martensitic phase to achieve a low-profile delivery configuration may be substantially recovered during a reverse phase transformation to austenite, such that the coils 18, 28 expand to a “remembered” (e.g., deployed) configuration at a treatment site in a vessel. Typically, recoverable strains of about 8-10% may be obtained from superelastic nickel-titanium alloys. The forward and reverse phase transformations may be driven by a change in stress (superelastic effect) and/or temperature (shape memory effect).

Slightly nickel-rich Nitinol alloys including, for example, about 51% Ni and about 49% Ti are known to be useful for medical devices which are superelastic at body temperature. In particular, alloys including 50.6-50.8% Ni and 49.2-49.4% Ti are considered to be medical grade Nitinol alloys and are suitable for the present coils 18, 28. The nickel-titanium alloy may include one or more additional alloying elements.

In a preferred embodiment, the wire 20 (i.e., primary coil 18) is made of nitinol or stainless steel and the wire 30 (i.e., secondary coil 28) is made of palladium. A primary coil 18 made of nitinol, for example, may provide many clinical advantages. After the nitinol tapered wire 20 is initially curled or coiled into the primary coil 18, it is effectively straightened-out in order to thread or slide the secondary coil 28 over it. Nitinol's super-elastic properties allow the tapered wire 20 to recover from the straightening strain and later return to its coiled primary shape.

Alternatively, the nitinol tapered wire 20 may be curled or coiled into the primary coil 18 and heat-set such that after it is effectively straightened for sliding the secondary coil 28 over it, the device 10 (i.e., the tapered wire 20 within the secondary coil 28) may be heated to a predetermined activating temperature to induce the shape-memory property of the nitinol tapered wire 20 and cause it to return to the coiled configuration (i.e., primary shape) of the primary coil 18, thus causing the secondary coil 28 to take on the primary shape of the primary coil 18.

In this embodiment, the device 10 may be stored in the straightened configuration for delivery to the interventionalist. As the device 10 is introduced into the body, body heat activates the shape-memory property of the nitinol tapered wire 20 within the secondary coil 28 and causes the tapered wire 20 to return to the primary shape of the primary coil 18, and thus causes the secondary coil 28 to take on the primary shape of the primary coil 18. The nitinol tapered wire 20 thus provides the secondary coil 28 with its secondary shape and variable stiffness due to the tapered diameter of the wire 20, therefore serving as an inner mandrel within the secondary coil 28.

In a particular embodiment, the proximal 32 and/or the distal end 34, of the secondary coil 28 includes a cap that may be soldered or welded to present to the coil 28 to provide a rounded or smooth surface, which will not catch on the interior surface of the guiding catheter or provide a source of trauma for the vasculature.

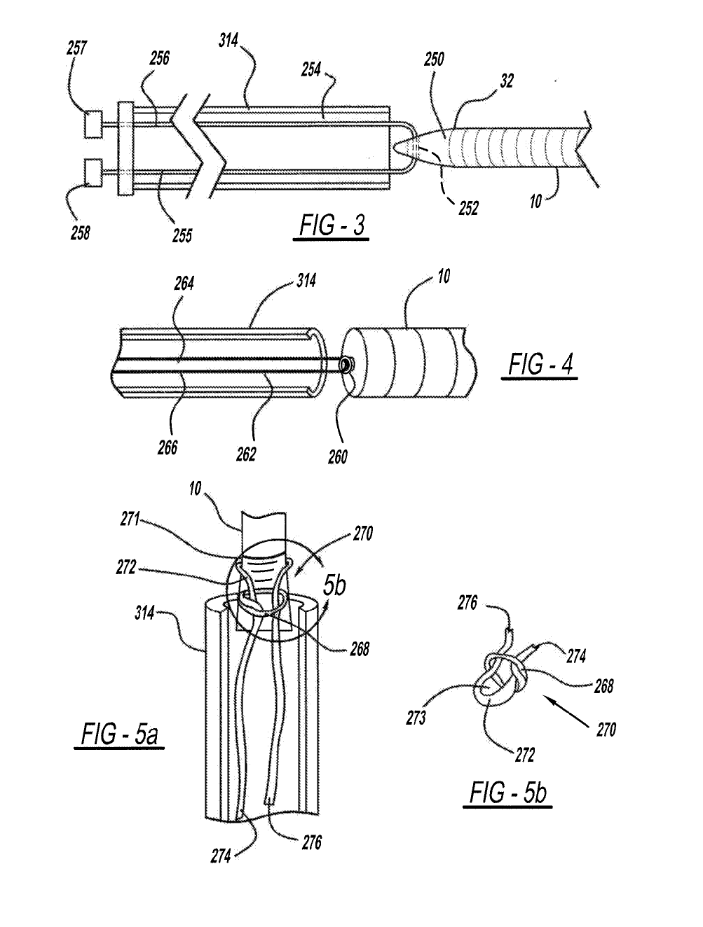

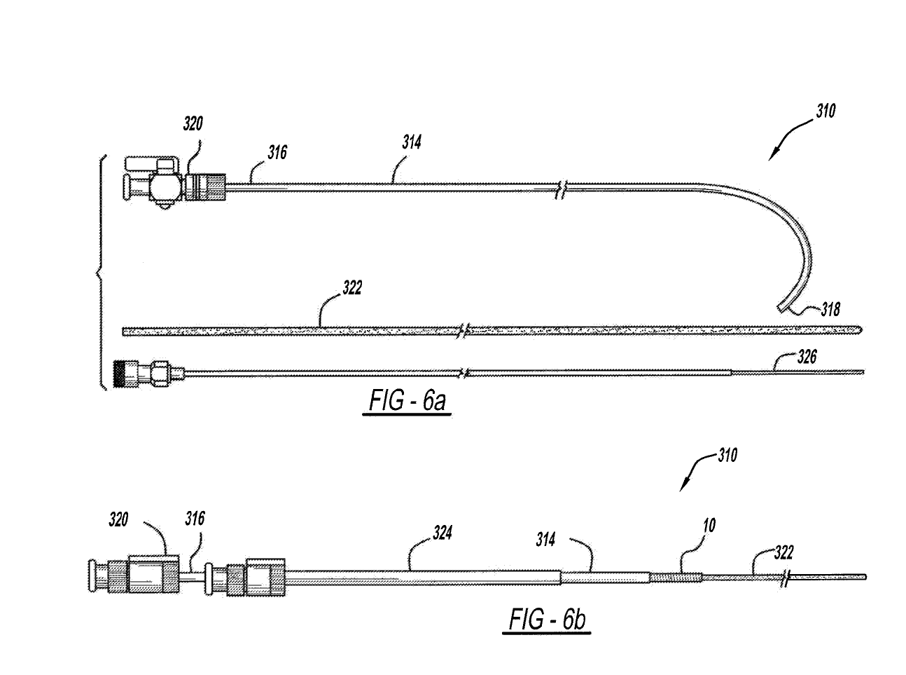

For example, as shown in FIG. 3, the proximal end 32 of the device 10 includes a cap 250 soldered, glued, or welded at the proximal end 32. The cap 250 is provided with an opening or hole 252 that extends laterally relative to the longitudinal axis 35 (FIGS. 1a and 1b) through which preferably a suture or an attacher 254, such as, for example, a monofilament, is threaded. The two extensions 255 and 256 of the suture 254 extend proximally through a catheter 314 and out of the proximal end of, for example, an embolization kit 310 (FIGS. 6a and 6b). Optionally, the respective ends of the extensions 256 and 258 may be attached to anchor tabs 257 and 258.

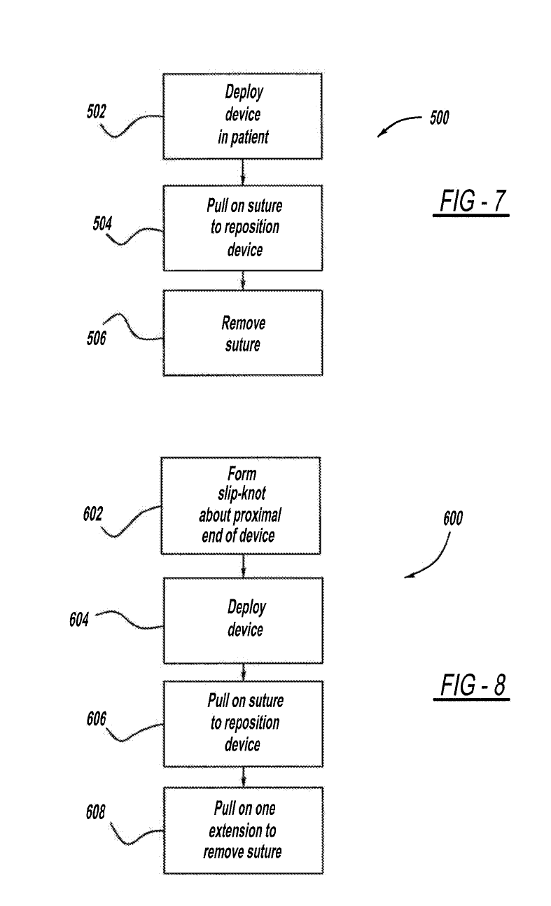

As such, as shown in FIG. 7, the physician may employ a process 500 to deploy and reposition the device 10 within the patient. In particular, after the device is deployed in step 502, the physician pulls on the two extensions 255 and 256 or the two tabs 257 and 258 to reposition the device 10.

Therefore, the physician can push the device 10 out the distal end of the catheter 314 with a wire and, if the device 10 is not in the desired position, then pull on the suture 254, and therefore the device 10, to reposition the device. During this process the device may potentially be pulled back into the catheter one or several times. When the device 10 is ready to be released, the physician, in step 506, pulls on one of the extensions to remove the suture 254 from the device 10. The suture 254 may be biodegradable so that it can be left in the patient's body. Additionally, the suture 254 may itself be thrombogenic in the vascular system to enhance the embolization.

In another form, as shown in FIG. 4, the device 10 includes an eyelet 260 through which the suture 254 is threaded. Again the extensions 256 and 255 extend proximally through the catheter 314 such that the deployment of the device 10 proceeds as described above with regard to the process 500.

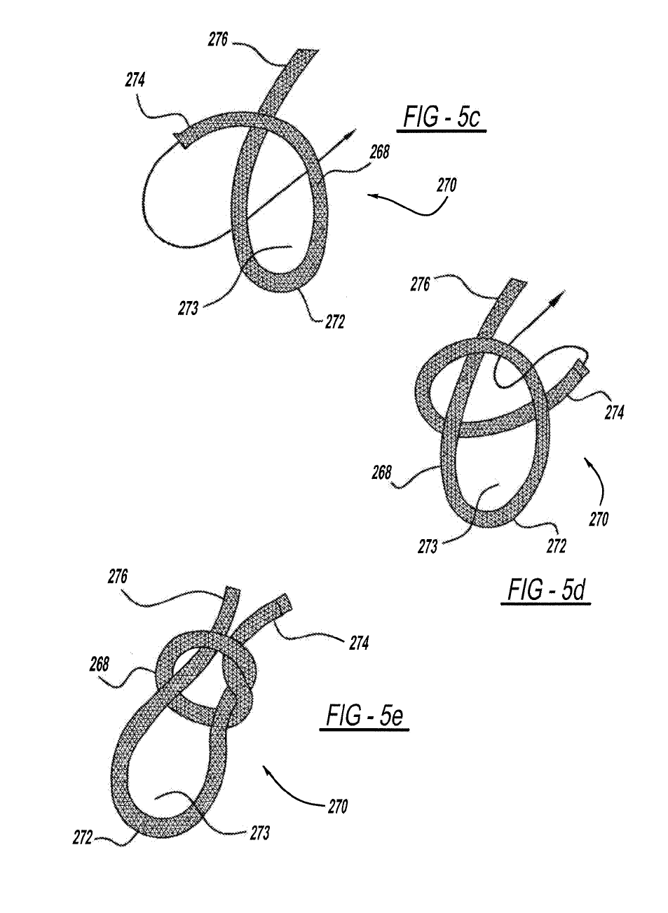

In yet another form, as shown in FIGS. 5a and 5b, the device 10 includes a cap 271 about which a suture 268 is formed or tied as a slip-knot 270 such that the cap 271 extends through an opening 273 of a loop portion 272 of the suture and two extensions 274 and 276 of the suture extend proximally through the catheter 314. Details of the formation of the slip knot 270 are shown in FIGS. 5c, 5d, and 5e. Initially (FIG. 5c), the extension 274 is brought under the extension 276 to form the loop portion 272. Then (FIG. 5d), the extension 274 is brought under and over the loop portion 272 through the opening 273. Finally (FIG. 5d), both extensions 274 and 276 are pulled to secure the slip-knot 270 about the cap 271.

Referring know to FIG. 8, a process 600 for the deployment of the device 10 with the slip-knot 270 is illustrated. The slip-knot 270 is formed or tied, as described above, about the cap 271 in step 602. In step 604, the device 10 is deployed, for example, with the embolization kit 310. If the device 10 is not in the correct position, the physician pulls on both extensions 274 and 276 (step 606) to retract the device 10 (potentially into the catheter 314) such that the device can be repositioned or redeployed. Finally, in step 608, the physician pulls on the extension 274 such that the extension 276 slides distally through the slip-knot 270 until the slip-knot unravels, allowing for the removal of the suture 268 from the patient. In a particular form, the suture 268 is made of polypropylene.

Turning now to FIGS. 6a and 6b, there is illustrated the embolization kit 310 which implements the occluding device 10 in accordance with one embodiment of the present invention. As shown, the kit 310 includes the inner catheter 314 preferably made from a soft, flexible material such as silicone or any other suitable material. Generally, the inner catheter 314 has a proximal end 316, a distal end 318, and a plastic adapter or hub 320 to receive apparatus to be advanced therethrough. In this embodiment, the inside diameter of the inner catheter may range between 0.014 and 0.027 inch. The kit 310 further includes a guide wire 322 which provides the guide catheter 324 (discussed in more detail below) a path during insertion of the guide catheter 324 within a body cavity. The size of the wire guide is based on the inside diameter of the guide catheter 324.

In this embodiment, the kit 310 further includes a polytetrafluoroethylene (PTFE) guide catheter or sheath 324 for percutaneously introducing the inner catheter 314 in a body vessel 14. Of course, any other suitable material may be used without falling beyond the scope or spirit of the present invention. The guide catheter 324 may have a size of about 4-French to 8-French and allows the inner catheter 314 to be inserted therethrough to a desired location in the body cavity. The guide catheter 324 receives the inner catheter 314 and provides stability of the inner catheter 314 at a desired location of the body cavity. For example, the guide catheter 324 may stay stationary within a common visceral artery, e.g., a common hepatic artery, and add stability to the inner catheter 314 as the inner catheter is advanced through the guide catheter to a point of occlusion in a connecting artery, e.g., the left or right hepatic artery.

When the distal end 318 of the inner catheter 314 is at the point of occlusion in the body cavity, the occluding device 10 is loaded at the proximal end 316 of the inner catheter 314 and is advanced through the inner catheter for deployment through the distal end 318. In this embodiment, a pushwire 326 is used to mechanically advance or push the occluding device 10 through the inner catheter 314. The size of the push wire used depends on the diameters of the inner catheter. As mentioned above, when the device 10 is deployed in the body vessel 14, the distal end 24 of the device 10 serves to hold the coil in place along the inner wall 16 of the body vessel 14. The proximal end 22 of the occluding device and the fibers 238 serve to occlude fluid flow by filling the lumen 12 of the body vessel 14.

In an alternative embodiment, an elongated releasing member made be used instead of a pushwire. The elongated releasing member is similar to the pushwire 326 in that it may be advanced through the inner catheter 314 to deploy the device 10 through the distal end 318. However, the elongated releasing member further includes a distal end configured for selectively engaging and/or disengaging with the device 10. Once the device 10 is deployed through the inner catheter 314, the elongated releasing member may be twisted or un-screwed to disengage the device 10 from the elongated releasing member, thus releasing the device 10 within the body vessel 14. Other suitable releasing devices known to those skilled in the art may also be used to advance and selectively deploy the occluding device 10 from the inner catheter 314.

As described earlier, depending upon the suture setup employed (FIG. 3, FIG. 4, or FIG. 5), the physician is able to pull on the extensions of the suture to reposition the device 10 or to retract the device 10 partially or completely into the catheter 314 for redeployment of the device 10.

It is to be understood that the embolization kit 310 described above is merely one example of a kit that may be used to deploy the occluding device in a body vessel. Of course, other kits, assemblies, and systems may be used to deploy any embodiment of the occluding device without falling beyond the scope or spirit of the present.

The aforementioned as well as other embodiments are within the following claims.

What is claimed is:

1. An occluding device apparatus comprising:
an embolization coil with a distal end and a proximal end, the proximal end having an opening; and
an attacher threaded through the opening at the proximal end of the embolization coil.
a delivery kit for delivery of the embolization coil in a body cavity, the kit comprising:
a guide catheter for percutaneous introduction of the embolization coil;
an inner catheter slidably disposed within the guide catheter during insertion, the inner catheter comprising a proximal end and a distal end, the inner catheter including a hub disposed adjacent the proximal end;
a guide wire slidably disposed within the inner catheter, the guide wire configured to provide a path during insertion thereof within a body cavity; and
a pushwire to advance the embolization coil through the inner catheter.

2. The occluding device of claim 1 wherein the attacher is a monofilament.

3. The occluding device of claim 1 further comprising a cap attached to the proximal end of the embolization coil, the opening extending through the cap laterally relative a longitudinal axis of the embolization coil.

4. The occluding device of claim 3 wherein the cap is fixed attached to the proximal end of the embolization coil.

5. The occluding device of claim 4 wherein the cap is fixedly attached to the proximal end of the embolization coil by one of weld, solder, and glue.

6. The occluding device of claim 3 wherein the cap is one of soldered, welded, and glued to the proximal end of the embolization coil.

7. The occluding device of claim 1 further comprising an eyelet attached to the proximal end of the embolization coil, the eyelet defining the opening through which the attacher is threaded.

8. The occluding device of claim 1 wherein the attacher has two extensions that extend proximally relative to the embolization coil through a catheter.

9. The occluding device of claim 8 wherein when tension is applied to the two extensions the embolization coil is retracted fully or partially into the catheter.

10. An occluding device comprising:
an embolization coil with a distal end and a proximal end; and
an attacher forming a slip-knot around the proximal end of the embolization coil.

11. The occluding device of claim 10 wherein the attacher is a monofilament.

12. The occluding device of claim 10 wherein the attacher is made of polypropylene.

13. The occluding device of claim 10 wherein the attacher has a first extension and a second extension.

14. The occluding device of claim 13 wherein the first and the second extensions extend proximally relative to the embolization coil through a catheter.

15. The occluding device of claim 14 wherein when tension is applied to both extensions the embolization coil is retracted fully or partially into the catheter.

16. The occluding device of claim 13 wherein when tension is applied to one of the first and the second extensions the other extension slips distally through the slip-knot such that the slip-knot unravels from the embolization coil.

17. A method of occluding fluid flow through a lumen of a body vessel, the method comprising:
deploying an embolization coil through a catheter into the lumen; and
pulling on an attacher connected to the embolization coil to retract the embolization coil fully or partially into the catheter; and
re-deploying the embolization coil in the lumen.

18. The method of claim 17 wherein the embolization coil has a distal end and a proximal end, the attacher being threaded through an opening at the proximal end of the embolization coil.

19. The method of claim 17 wherein the attacher is tied as a slip-knot about the proximal end of the embolization coil.

20. The method of claim 19 wherein the attacher has a first extension and a second extension and wherein pulling on the first or the second extensions causes the other extension to slip distally through the slip-knot such that the slip-knot unravels from the embolization coil.
